(19) United States
(12) Patent Application Publication
(10) Pub. No.: US 2020/0214371 A1
(43) Pub. Date: Jul. 9, 2020

(54) GARMENT FOR USE WITH STOMAS

(71) Applicant: Cynthia Apelt, Strongsville, OH (US)

(72) Inventor: Cynthia Apelt, Strongsville, OH (US)

(21) Appl. No.: 16/737,080

(22) Filed: Jan. 8, 2020

Related U.S. Application Data
(60) Provisional application No. 62/790,264, filed on Jan. 9, 2019.

(51) Int. Cl. A41D 13/12; A61F 5/449

(52) U.S. Cl. CPC A41D 13/1245; A41D 2600/00; A61F 5/449

(57) ABSTRACT

Disclosed herein are form-fitting garments configured to hold an ostomy pouch close to the body via a shaped interior pocket creating a barrier between the pouch and skin of an individual. The garment may include a plurality of pockets such that an unused pocket may serve to hold additional supplies. The garment is preferably configured to have a maximum stretch in the horizontal direction allowing for the expansion of the pouch while having minimal stretching in the vertical direction to prevent the interior pocket and garment from sagging under the weight of the pouch.

### CROSS REFERENCE TO RELATED APPLICATION

This application claims priority to U.S. Provisional Patent Application Ser. No. 62/790,264, filed Jan. 9, 2019, the entirety of which is fully incorporated herein by reference.

### BACKGROUND

The present exemplary embodiment relates to form-fitting garments for supporting medical devices and/or appliances. It finds particular application in conjunction with supporting ostomy pouches and will be described with particular reference thereto. However, it is to be appreciated that the present exemplary embodiment is also amenable to other like applications.

An ostomy pouch is a medical device configured to collect human waste from a stoma. The pouches are commonly associated with colostomies, ileostomies, and urostomies. The ostomy pouch is attached mechanically or with an adhesive, creating an airtight seal about an individual's stoma. While the design of the ostomy pouches has improved, wearing an ostomy pouch affects the contours of the body of an individual and the profile of the ostomy pouch may be noticeable when wearing form-fitting clothing. Furthermore, as a ostomy pouch fills with waste, the weight of the pouch increases and may provide stress on the stoma. Current garments are insufficient in providing form-fitting styles with ample support for those individuals with stomas.

Thus, there is a need in the art to provide form-fitting garments, which may hold and support an ostomy pouch, wherein the weight of the ostomy pouch is distributed to an individual's shoulders relieving stress on the ostomy.

### BRIEF DESCRIPTION

Various details of the present disclosure are hereinafter summarized to provide a basic understanding. This summary is not an extensive overview of the disclosure and is neither intended to identify certain elements of the disclosure, nor to delineate scope thereof. Rather, the primary purpose of this summary is to present some concepts of the disclosure in a simplified form prior to the more detailed description that is presented hereinafter.

Disclosed herein are form-fitting garments configured to hold an ostomy pouch close to the body via a shaped interior pocket creating a barrier between the pouch and skin of an individual. The garment may include at least one interior pocket configured to receive and support an ostomy pouch. The garment is configured to have a maximum stretch in the horizontal direction allowing for the expansion of the filling ostomy pouch while having minimal stretching in the vertical direction to prevent the interior pocket and garment from sagging under the weight of the ostomy pouch. The garment includes at least one shoulder support for distributing the weight of the ostomy pouch to at least one shoulder of the individual.

Specifically, in accordance with one embodiment of the disclosure, an ostomy garment configured to be worn by an individual is disclosed. The ostomy garment includes an interior surface having at least one shaped pocket located in a lower front position of the garment and configured to receive an ostomy pouch. At least one shoulder support for distributing a weight of the ostomy pouch to at least one shoulder of the individual is also included. Finally, the ostomy garment is a form-fitting garment configured to adapt to a body contour of the individual.

According to one aspect of the embodiments disclosed herein, the ostomy garment includes a 1-way or 2-way stretchable material. The 1-way stretchable material can stretch in a horizontal or vertical direction. The 2-way stretchable material is configured to stretch in a horizontal direction, i.e., side to side or in a vertical direction, i.e., top to bottom. In accordance with other aspects, the 2-way stretchable material is configured with a greater capacity for stretching in the horizontal direction than in a vertical direction. In some particular embodiments, the 1-way or 2-way stretchable material is spandex, elastane, or neoprene.

According to another aspect of the disclosure, the garment and/or the pocket is stitched to have minimal stretching.

In accordance with another aspect of the presently disclosed embodiments, the ostomy garment further includes a front fastener. The front fastener can extend from a top hem of the garment to a bottom hem of the garment.

According to other aspects of the disclosure, the at least one pocket of the ostomy garment is rectangularly shaped. According to additional aspects, the at least one pocket of the ostomy garment is wedge shaped.

According to further aspects of the embodiments disclosed herein, the ostomy garment includes a liner held within an interior surface of the at least one pocket. The liner can be removable from the at least one pocket. Moreover, the liner can be held within the at least one pocket by a snap fastener, a zipper, a sliding fastener, a twist tie, a clip fastener, or a hook and loop fastener.

In accordance with another aspect of the presently disclosed embodiments, the at least one pocket of the ostomy garment includes a first pocket located in the lower front position on a left side of the garment and a second pocket located in the lower front position on a right side of the garment. The first and second pockets can have a rectangular shape or a wedge shape.

According to other aspects of the disclosure, the form-fitting garment is at least one of a tank-top, a camisole, or lingerie.

In accordance with another embodiment of the disclosure, a method for discreetly using an ostomy pouch with a garment worn by an individual is disclosed. The method includes providing a stretchable material for making the garment and a front fastener for closing and opening the garment, the stretchable material being configured to stretch in at least a horizontal direction; forming at least one shaped pocket in a lower front position of an interior surface of the garment; receiving and supporting the ostomy pouch within the at least one shaped pocket; and, manipulating the front fastener such that the garment is in a form-fitting position with the ostomy pouch being held securely to the individual.

According to one aspect of the embodiments disclosed herein, the method for discreetly using an ostomy pouch with a garment further includes making the garment from the stretchable material such that the garment has a greater capacity for stretching in the horizontal direction than in a vertical direction.

In accordance with other aspects of the presently disclosed embodiments, the method for discreetly using an ostomy pouch with a garment further includes forming the at least one shaped pocket to have a rectangular or a wedge shape.

In accordance with an additional embodiment of the disclosure, an ostomy garment configured to be worn by an individual is disclosed which includes an interior surface having at least one shaped pocket located in a lower front position of the garment. The at least one shaped pocket is configured to receive an ostomy pouch. A 2-way stretchable material configured with a greater capacity for stretching in a horizontal direction than in a vertical direction is also included. Furthermore, the ostomy garment also includes a front fastener extending from a top hem of the garment to a bottom hem of the garment. The front fastener is configured to be manipulated such that the garment is in a form-fitting position and the ostomy pouch is held securely to the individual.

### BRIEF DESCRIPTION OF THE DRAWINGS

The following is a brief description of the drawings, which are presented for the purposes of illustrating the exemplary embodiments disclosed herein and not for the purposes of limiting the same.

FIG. 1 illustrates an exemplary ostomy garment in accordance with the present disclosure.

FIG. 2A illustrates exemplary ostomy garment pockets in accordance with the present disclosure.

FIG. 2B illustrates exemplary ostomy pouches in accordance with the present disclosure.

FIG. 3A illustrates other exemplary ostomy garment pockets in accordance with the present disclosure.

FIG. 3B illustrates exemplary ostomy pouches in accordance with the present disclosure.

FIG. 4 illustrates an exemplary pocket liner in accordance with the present disclosure.

FIG. 5 illustrates exemplary ostomy lingerie in accordance with the° present disclosure.

FIG. 6 illustrates exemplary ostomy tank-top in accordance with the present disclosure.

FIG. 7 illustrates an exemplary pull over ostomy camisole in accordance with the present disclosure.

### DETAILED DESCRIPTION

A more complete understanding of the articles/devices, processes and components disclosed herein can be obtained by reference to the accompanying drawings. These figures are merely schematic representations based on convenience and the ease of demonstrating the present disclosure, and are, therefore, not intended to indicate relative size and dimensions of the devices or components thereof and/or to define or limit the scope of the exemplary embodiments.

Although specific terms are used in the following description for the sake of clarity, these terms are intended to refer only to the particular structure of the embodiments selected for illustration in the drawings and are not intended to define or limit the scope of the disclosure. In the drawings and the following description below, it is to be understood that like numeric designations refer to components of like function.

The singular forms “a,” “an,” and “the” include plural referents unless the context clearly dictates otherwise.

As used in the specification and in the claims, the term “comprising” may include the embodiments “consisting of” and “consisting essentially of.” The terms “comprise(s),” “include(s),” “having,” “has,” “can,” “contain(s),” and variants thereof, as used herein, are intended to be open-ended transitional phrases, terms, or words that require the presence of the named ingredients/steps and permit the presence of other ingredients/steps. However, such description should be construed as also describing compositions or processes as “consisting of” and “consisting essentially of” the enumerated ingredients/steps, which allows the presence of only the named ingredients/steps, along with any unavoidable impurities that might result therefrom, and excludes other ingredients/steps.

Numerical values in the specification and claims of this application should be understood to include numerical values which are the same when reduced to the same number of significant figures and numerical values which differ from the stated value by less than the experimental error of conventional measurement technique of the type described in the present application to determine the value.

All ranges disclosed herein are inclusive of the recited endpoint and independently combinable (for example, the range of “from 2 grams to 10 grams” is inclusive of the endpoints, 2 grams and 10 grams, and all the intermediate values).

The terms “about” and “approximately” can be used to include any numerical value that can vary without changing the basic function of that value. When used with a range, “about” and “approximately” also disclose the range defined by the absolute values of the two endpoints, e.g. “about 2 to about 4” also discloses the range “from 2 to 4.” Generally, the terms “about” and “approximately” may refer to plus or minus 10% of the indicated number.

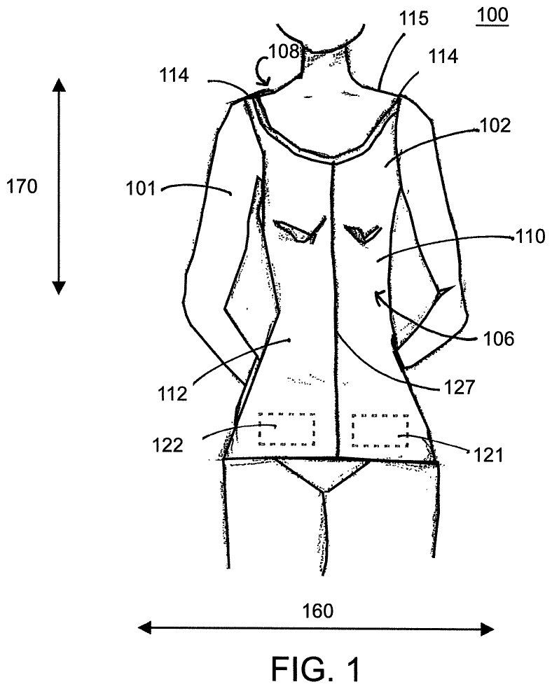

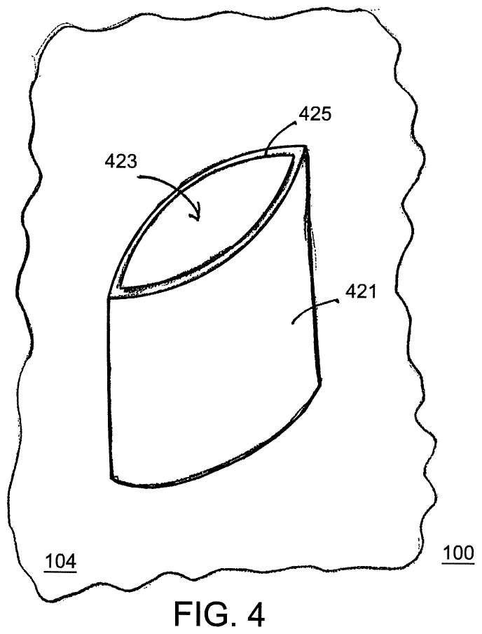

In accordance with the present disclosure and with reference to FIG. 1, an ostomy garment 100 is provided. The ostomy garment 100 includes an exterior surface 102, an interior surface 104 (shown in FIG. 4), a front 106, a back 108, a left side 110 and a right side 112. When the ostomy garment 100 is worn by an individual 101, the interior surface 104 of the ostomy garment 100 is in contact with the body of an individual 101. The ostomy garment 100 also includes at least one shoulder support 114 configured to support the garment 100 on the shoulders 115 of the individual 101. Thus, any weight imparted to the garment 100 (for example, the weight of a filling ostomy pouch) is distributed to the at least one shoulder support 114 and shoulders 115.

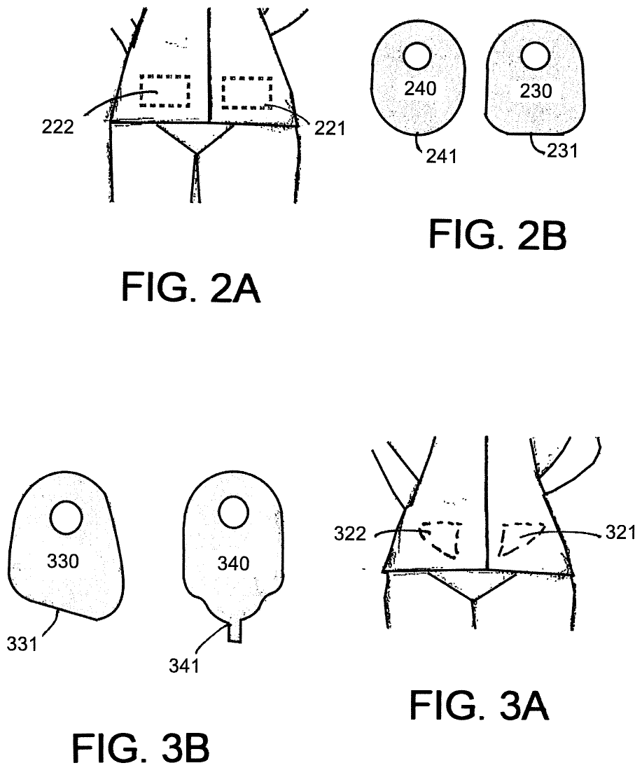

The interior surface 104 includes at least one pocket 121 in a lower front 106 region of the ostomy garment 100 on either the left side 110 or right side 112. In some embodiments and as illustrated in FIGS. 1- 3, the interior surface 104 includes a set of pockets, a first pocket 121 located about the lower front 106 left side 110 and a second pocket 122 located about the lower front 106 right side 112 of the garment 100. The first and second pockets 121 and 122, are shaped to receive and support an ostomy pouch. That is, an ostomy pouch, such as pouches 230, 240, 330 and 340 of FIGS. 2 and 3, is able to be inserted into a shaped pocket after the garment 100 is dressed on the individual 101. It is understood that ostomy pouches are available in a limited number of shapes, thus, the variations of the design for the first and second pockets 121 and 122 are also limited.

It is to be appreciated that when an ostomy pouch is worn inside a pocket 121, 122, the exterior surface of the ostomy pouch is in contact with the interior (423 of FIG. 4) of the pocket 121, 122, and not with the skin of the individual 101. That is the inside of the pocket 121, 122 creates a barrier between the pouch and the skin which helps protect the skin and improve the overall comfort of the ostomy pouch and garment 100 combination. The barrier property of the pocket also reduces the chance for the development of yeast infections. There are many variations of the pocket sizes so as to accommodate the various types and sizes of bags. Most pockets will extend to within one quarter of an inch of the lower hem of the garment. Since the garments will range from XXS to XXXL, some garments may be disproportionally long to accommodate the bag.

One embodiment is a pocket that extends horizontally from the front seam around to the side seam to accommodate people who prefer to wear their bags at an angle. The pocket shapes are similar to the shape of the bag, be it a closed end pouch, a drainable pouch with a tail clip, a drainable pouch with a clipless closure, etc.

Furthermore, with the ostomy pouch secured in place within a pocket 121, 122 of the form-fitting garment 100, the chance for the ostomy pouch to loosen, leak, or fall off is lowered compared to wearing loose fitting clothing over the ostomy pouch. That is, the ostomy pouch is protected by the garment 100 from excessive movement.

In some embodiments and illustrated in FIG. 2A, the pockets 221 and 222 are substantially rectangular in shape. These pockets 221, 222 may optimally support ostomy pouches 230 and 240 (FIG. 2B) having flat bottom edges 231 or rounded bottom edges 241 respectively. The pockets 221, 222 may also accommodate pouches having other shapes and sizes.

In some embodiments and illustrated in FIG. 3A, the pockets 321 and 322 are substantially wedged shaped. Referring to FIG. 3B, these pockets may optimally support ostomy pouches 330 having angled bottom edges 331. The wedged shaped pockets 321 and 322 may also accommodate an ostomy pouch 340 having a drain portion 341.

In some embodiments, and with reference to FIG. 4, an interior 423 of a pocket 421 includes a liner 425. In some embodiments, the liner 425 is removable from the interior 423 of the pocket 421 and may be disposed of and replaced. In more particular embodiments, the liner 425 also includes a zip-top seal. In yet still more particular embodiments, the liner 425 is made of a plastic material. Thus, the liner 425 of the pocket 421 may also be used as an odor proof bag for discreet disposal of an ostomy pouch. The pocket may hold an optional insertable liner with either a snap shut, zip shut, slide shut, twist tie shut, or clip shut opening. The insert would be a safeguard against embarrassment and a means of enclosing a bag or its contents for later disposal.

Some embodiments will have a front or side closure which could be either full or partial length. The fastener could be a zipper from the top, a zipper from the bottom hem, a zipper that works from either direction, or a partial zipper; it could be an invisible zipper or another type of zipper. Any opening could be closed by Velcro, hook and eye, snap, ties or virtually any type of suitable closure. Some embodiments will provide an access point to the pocket or pockets from the outside or from both the outside and the inside.

With reference back to FIG. 1, the ostomy garment 100 is configured to be a form-fitting garment. That is, the garment 100 tightly follows the counters of the upper body of an individual 101. The ostomy garment 100 accomplishes its form-fitting feature through its fabric composition. That is, the material of the garment 100 is a stretchable and/or elastic material. In some embodiments, the material of the garment 100 comprises synthetic fibers. Nonlimiting examples of such synthetic fibers include spandex, elastane, and neoprene.

In some embodiments, and with continued reference to FIG. 1, the material of the ostomy garment 100 is configured to have a 1-way or a 2-way stretch. Any variety of fabric which is soft and flexible can be used for the garment. A 1-way stretch can be stretched in a horizontal or vertical direction and preferably returns to its original shape. A 2-way stretch fabric is one that stretches horizontally (side to side) or vertically (top to bottom) and recovers the stretch (returns to its original shape). Preferably, the ostomy garment 100 is configured to have a greater capability of stretching in the horizontal direction 160 than in the vertical direction 170. The stretch in the horizontal direction 160 while creating a form-fitting appearance also allows for the expansion of an ostomy pouch worn by an individual 101. With minimal stretching allowed in the vertical direction 170, undesirable sagging of the ostomy garment 100, experiencing the weight of a filling ostomy pouch, is prevented. Another embodiment has stitching which results in minimal stretching of the garment and the pockets (121,122).

In some embodiments, the ostomy garment 100 includes a front fastener 127 for closing and opening the garment 100, for example and without limitation a plurality of buttons and/or a zipper. Other fasteners such as velcro could also be used. This allows the individual 101 wearing the garment 100 to have easier access to the at least one interior pocket 121, 122, for insertion of the ostomy pouch. With the ostomy pouch inserted, the fastener 127 can be manipulated such that the garment 100 is in a snug, form-fitting position holding the pouch securely to the body of the individual 101.

The present disclosure is further illustrated in the following non-limiting working examples, it is being understood that these examples are intended to be illustrative only and that the disclosure is not intended to be limited to the materials, conditions, process parameters and the like recited herein. In addition to the physical benefits, a level of increased comfort for ostomates will result when using the garments and an increase in the willingness and acceptance of the user of the surgical procedure when the user is shown the option of the garment. The garment will help to make the usage of an ostomy pouch more discreet, contained, comfortable and secure.

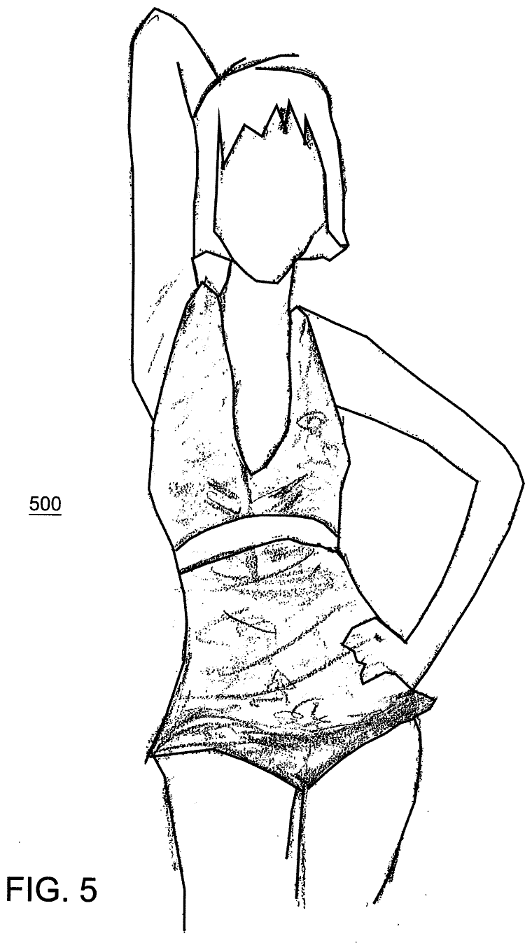

FIG. 5 illustrates a form-fitting lingerie garment 500 incorporating the interior pockets and 2-way stretchable fabric described with respect to FIGS. 1-4.

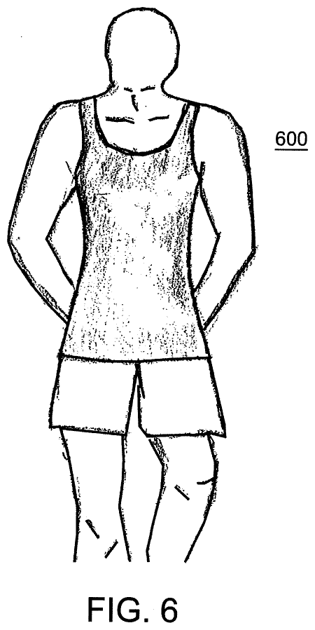

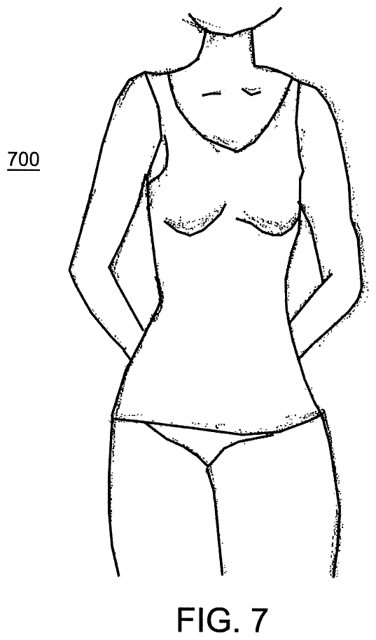

FIG. 6 illustrates a form-fitting tank top garment 600 incorporating the interior pockets and 2-way stretchable fabric described with respect to FIGS. 1-4.

FIG. 7 illustrates a form-fitting pullover camisole 700 incorporating the interior pockets and 2-way stretchable fabric described with respect to FIGS. 1-4.

The exemplary embodiment has been described with reference to the preferred embodiments. Obviously, modifications and alterations will occur to others upon reading and understanding the preceding detailed description. It is intended that the exemplary embodiment be construed as including all such modifications and alterations insofar as they come within the scope of the appended claims or the equivalents thereof.

To aid the Patent Office and any readers of this application and any resulting patent in interpreting the claims appended hereto, applicants do not intend any of the appended claims or claim elements to invoke 35 U.S.C. 112(f) unless the words “means for” or “step for” are explicitly used in the particular claim.

1. An ostomy garment configured to be worn by an individual comprising,
an interior surface having at least one shaped pocket located in a lower front position of the garment and configured to receive an ostomy pouch; and
at least one shoulder support for distributing a weight of the ostomy pouch to at least one shoulder of the individual,
wherein the ostomy garment is a form-fitting garment configured to adapt to a body contour of the individual.

2. The ostomy garment according to claim 1, further comprising a 2-way stretchable material.

3. The ostomy garment according to claim 2, wherein the 2-way stretchable material is configured to stretch in a horizontal direction.

4. The ostomy garment according to claim 2, wherein the 2-way stretchable material is configured with a greater capacity for stretching in the horizontal direction than in a vertical direction.

5. The ostomy garment according to claim 2, wherein the 2-way stretchable material is spandex, elastane, or neoprene.

6. The ostomy garment of claim 1, further comprising a 1-way stretchable material which stretches horizontally or vertically.

7. The ostomy garment according to claim 1, further comprising a front fastener.

8. The ostomy garment according to claim 7, wherein the front fastener extends from a top hem of the garment to a bottom hem of the garment

9. The ostomy garment according to claim 1, wherein the at least one pocket is rectangularly shaped.

10. The ostomy garment according to claim 1, wherein the at least one pocket is wedge shaped.

11. The ostomy garment according to claim 1, further comprising a liner held within an interior surface of the at least one pocket.

12. The ostomy garment according to claim 11, wherein the liner is configured to be removed from the at least one pocket.

13. The ostomy garment according to claim 11, wherein the liner is held within the at least one pocket by a snap fastener, a zipper, a sliding fastener, a twist tie, a clip fastener, or a hook and loop fastener.

14. The ostomy garment according to claim 1, wherein the at least one pocket further comprises a first pocket located in the lower front position on a left side of the garment and a second pocket located in the lower front position on a right side of the garment.

15. The ostomy garment according to claim 14, wherein the first and second pockets have a rectangular shape or a wedge shape.

16. The ostomy garment according to claim 1, wherein the form-fitting garment is at least one of a tank-top, a camisole, or lingerie.

17. A method for discreetly using an ostomy pouch with a garment worn by an individual comprising:
providing a stretchable material for making the garment and a front fastener for closing and opening the garment, the stretchable material being configured to stretch in at least a horizontal direction;
forming at least one shaped pocket in a lower front position of an interior surface of the garment;
receiving and supporting the ostomy pouch within the at least one shaped pocket; and,
manipulating the front fastener such that the garment is in a form-fitting position with the ostomy pouch being held securely to the individual.

18. The method of claim 17, further comprising making the garment from the stretchable material such that the garment has a greater capacity for stretching in the horizontal direction than in a vertical direction.

19. The method of claim 17, further comprising forming the at least one shaped pocket to have a rectangular shape.

20. The method of claim 17, further comprising forming the at least one shaped pocket to have a wedge shape.

21. An ostomy garment configured to be worn by an individual comprising,
an interior surface having at least one shaped pocket located in a lower front position of the garment and configured to receive an ostomy pouch;
a 1-way or 2-way stretchable material configured to stretch in a horizontal direction or a vertical direction; and,
a front fastener extending from a top hem of the garment to a bottom hem of the garment, the front fastener configured to be manipulated such that the garment is in a form-fitting position and the ostomy pouch is held securely to the individual.
